(19) United States
(12) Patent Application Publication
(10) Pub. No.: US 2004/0126811 A1
(43) Pub. Date: Jul. 1, 2004

(54) Helicobacter pylori sialic acid binding adhesin, saba and saba-gene

(76) Inventors: Thomas Boren, Umea (SE); Lennart Hammarstrom, Huddinge (SE)

Correspondence Address: Bacon & Thomas, 4th Floor, 625 Slaters Lane, Alexandria, VA, 22314-1176, US

(21) Appl. No.: 10/467,336

(22) PCT Filed: Feb. 21, 2002
(86) PCT No.: PCT/SE02/00301

(51) Int. Cl. C12Q 1/68; G01N 33/53

(52) U.S. Cl. 435/7.1

(57) ABSTRACT

An isolated Helicobacter pylori protein binding to sialyl-Lewis x antigen and having an approximate molecular weight of 66 kDa and comprising the amino acid sequences SEQ ID NO: 1, SEQ ID NO: 2, SEQ ID NO: 3, SEQ ID NO: 4, and sialyl-Lewis x antigen-binding H. pylori alleles of the protein, recombinant forms of the protein, such as a protein having the amino acid sequence SEQ ID NO: 5, or the protein alleles, and sialyl-Lewis x antigen binding portions of the proteins, are disclosed. The protein or portion of protein maybe used as a medicament or diagnostic antigen, and can be used in a method of determining the presence of sialyl-Lewis x antigen-binding H. pylori bacteria in a biological sample. Further, a DNA molecule encoding the protein or portion of protein, a vector comprising the DNA molecule, and a host transformed with the vector are comprised by the disclosure. Additionally, a method of determining the presence of sialyl-Lewis x or related carbohydrate structures in a sample, is described. This method has a wide range of different applications.

The present invention relates to a Helicobacter pylori Sialic acid binding Adhesin, SabA and sabA—gene. In particular, the invention relates to an isolated Helicobacter pylori protein binding to sialyl-Lewis x antigen and having an approximate molecular weight of 66 kDa. The protein, or a sialyl-Lewis x antigen binding portion of the protein, may be used as a medicament or diagnostic antigen, and it can be used in a method of determining the presence of sialyl-Lewis x antigen-binding H. pylori bacteria in a biological sample. The invention comprises also a DNA molecule encoding the protein or a sialyl-Lewis x antigen binding portion of the protein, a vector comprising the DNA molecule, and a host transformed with the vector.

### BACKGROUND

Helicobacter pylori is considered the causative agent of chronic active gastritis and peptic ulcer disease (Marshall and Warren, 1984), and is also correlated to development of gastric cancer (Parsonnet, 1998). H. pylori colonizes the human gastric epithelial lining and the mucus layer of primates and humans. For adherence, bacteria express attachment molecules (adhesins) that bind specifically to cell surface proteins and glycoconjugates i.e., the receptors (Hultgren et al., 1993). Thus, the adhesins will target the infection to a limited number of hosts, tissues and cell lineages (Karlsson, 1998).

We have previously demonstrated H. pylori adherence to the fucosylated blood group antigen H1 and Lewis b (Borén et al., 1993). The H-antigen is typically found on erythrocytes defining the O phenotype in the ABO blood group system, but the fucosylated histo-blood group antigens are also expressed on the epithelial cell surfaces along the gastrointestinal tract (Clausen et al., 1989). Individuals of blood group O phenotype are common among patients suffering from peptic ulcer disease (discussed in Borén et al., 1994). Recently we found that the Lewis b antigen binding property is prevalent among the virulent strains that carry the cag-Pathogenicity Island and the vacuolating cytotoxin i.e., triple-positive strains. We therefore propose that Lewis b antigen mediated adherence of H. pylori plays a critical role for development of severe disease (Gerhard et al., 1999). Adherence of H. pylori to the gastric epithelial lining was recently demonstrated in the transgenic Lewis b mouse expressing human α1,¾ fucosyltransferase (Falk et al., 1995). Challenge experiments suggest that H. pylori adherence mediated by the Lewis b antigen activate inflammatory responses (Guruge et al., 1998).

In order to identify the Lewis b antigen binding H. pylori adhesin we developed the Retagging-technique (Ilver-Arnqvist et al., 1998). The blood group antigen binding adhesin, BabA, belongs to a family of outer membrane proteins (Tomb et al., 1997). We have previously shown that a babA-mutant strain although totally devoid of Lewis b antigen binding propeties, still adheres avidly to the human gastric epithelial lining (WO 00/56343). We have also previously identified the sialyl-dimeric-Lewis x glycosphingolipid to which the babA-mutant strain adheres with high affinity (WO 00/56343).

### DESCRIPTION OF THE INVENTION

The present invention provides a sialic acid binding adhesin, SabA, binding the sialyl-Lewis x antigen. SabA was identified and purified from the Helicobacter pylori babA-mutant by the Retagging-technique and it binds to the sialyl dimeric-Lewis x glycosphingolipid to which the babA-mutant strain adheres (WO 00/56343). Our new results suggest a flexible adaptation of bacterial adherence properties by alternative adherence modes and adhesins, to meet various inflammatory responses, such as defensive shifts in the detailed glycosylation patterns of the gastric mucosa and the epithelial lining, during the course of chronic infectious disease.

The present invention is particularly directed to an isolated Helicobacter pylori protein binding to sialyl-Lewis x antigen and having an approximate molecular weight of 66 kDa (i.e. the actual molecular weight may be up to 10 % higher) and comprising the amino acid sequences

| | |
|---|---|
| SEQ ID NO:1, | QSIQNANNIELVNSSLNYLK, |
| SEQ ID NO:2, | IPTINTNYYSFLGTK, |
| SEQ ID NO:3, | YYGFFDYNHGYIK, and |
| SEQ ID NO:4, | DIYAFAQNQK, |

and sialyl-Lewis x antigen-binding H. pylori alleles of the protein and recombinant forms of the protein, such as SEQ ID NO: 5, or the protein alleles, or sialyl-Lewis x antigen binding portion of the proteins. The recombinant proteins are thus expressed from a gene encoding the sialyl-Lewis x antigen-binding protein or the alleles.

The alleles of the protein may have an amino acid sequence that differs from the isolated H.pylori protein with up to 15%, normally about 10% or less, such as 5%, but they shall have sialyl-Lewis x antigen-binding properties to be comprised by the present invention.

The recombinant forms of the protein may have the amino acid sequence of the full length isolated protein or its alleles or may have an amino acid sequence that corresponds to a sialyl-Lewis x antigen binding fragment of the isolated protein or one of its alleles or an optimized amino acid sequence with regard to production requirements and/or immunizing properties.

The invention is also directed to the use of a protein or a sialyl-Lewis x antigen binding portion of a protein comprised by the invention for use as a medicament. The medicament may be used for inhibition of H. pylori binding to human tissues since the proteins or sialyl-Lewis x antigen parts of the proteins of the invention bind to human or animal glycoconjugates presented on patient's tissues. Further, the medicament may be a therapeutic or prophylactic vaccine against Helicobacter pylori infection, wherein the protein is an active ingredient, optionally together with other active ingredients, such as other Helicobacter pylori antigenic proteins. The formulations of the medicaments or vaccines of the invention will be decided by the manufacturer using Good Manufacturing Procedure accepted by the medical authorities. The doses administered to patients will be decided by the patient's physician based on recommendations from the manufacturer.

The invention is further directed to a diagnostic antigen for the immunological determination, in a biological sample, of antibodies against sialyl-Lewis x antigen-binding protein, wherein the diagnostic antigen is an optionally labeled protein or a sialyl-Lewis x antigen binding portion of a protein comprised by the present invention. Examples of the biological sample are a biopsy, blood or plasma sample, and examples of immunological determinations are ELISA-assays and RIA-assays. Thus, the proteins and the sialyl-Lewis x antigen-binding portions of the proteins of the invention may be conjugated to a reporter molecule, such as a fluorescent marker, radiolabelling or an enzyme producing a detectable signal or biotin or other affinity tag to enable recognition of the labeled molecule of the invention.

Another aspect of the invention is directed to a method of determining the presence of sialyl-Lewis x antigen-binding H. pylori bacteria in a biological sample, which comprises an immunological determination of the presence of antibodies binding to an optionally labeled protein comprised by the invention. An example of the biological sample is a biopsy sample.

The invention is also directed to a DNA molecule encoding a protein or a sialyl-Lewis x antigen binding portion of a protein according to the invention, a vector comprising the DNA molecule, and a host transformed with the vector. The DNA molecule may be isolated or synthetic and will only code for a protein or part of the protein of the invention. The vector may comprise, in addition to the DNA molecule of the invention, genes or gene fragments for the construction of fusion proteins, e.g. recombinant SabA-fusion proteins for different purposes. The vector of the invention is preferably a plasmid, and the host is preferably a microorganism. The DNA molecule, the vector and the host are useful in the production of a recombinant protein or a sialyl-Lewis x antigen binding portion of a protein comprised by the invention. Methods of producing recombinant proteins are well-known to a man skilled in the art of biotechnology.

Yet another aspect of the invention is directed to a method of determining the presence of sialyl-Lewis x or related carbohydrate structures in a sample, comprising bringing the sample into contact with an optionally labelled protein or sialyl-Lewis x antigen binding portion of a protein according to claim 1 or 2, allowing binding of the protein or sialyl-Lewis x antigen binding portion of the protein according to claim 1 or 2 to the carbohydrate structure and determining the presence of sialyl-Lewis x or related carbohydrate structures in the sample by determining

a) the occurrence of the binding, or

b) the absence of binding in case an analyte inhibiting the binding is present.

The binding of the proteins and the sialyl-Lewis x antigen-binding portions of the proteins of the invention or their labeled molecules to carbohydrate structures, in particular sialyl-Lewis x and related carbohydrates, can be used for several applications, such as diagnostic purposes, for protein purification, screening of substances which bind to proteins and the sialyl-Lewis x antigen-binding portions of the proteins of the invention including human and animal glycoconjugates, and to detect receptors for H. pylori or pathologic changes of the tissue. Preferably the tissue or sample or preparation of tissue is from human gastric tissue or from human tumor tissue. Therefore, the proteins and the sialyl-Lewis x antigen-binding portions of the proteins of the invention can be used in a method of diagnosing a disease, preferably a gastric disease, cancer or an inflammatory disease.

The proteins and the sialyl-Lewis x antigen-binding portions of the proteins of the invention can be used in assays to determine, e.g. by measurement, the binding to the proteins and the sialyl-Lewis x antigen-binding portions of the proteins of the invention of carbohydrates, such as sialyl-Lewis x and other carbohydrate substances or carbohydrate analog substances. Such assays may measure a) direct binding of the proteins and the sialyl-Lewis x antigen-binding portions of the proteins of the invention to carbohydrates or b) inhibition by the analyte of binding of a proteins and the sialyl-Lewis x antigen-binding portions of the proteins of the invention to a carbohydrate ligand. The assays may be performed in solution by use of e.g. NMR or in solid phase in numerous formats in which the proteins and the sialyl-Lewis x antigen-birding portions of the proteins of the invention or their ligands can be immobilized. The assays to determine binding to the proteins and the sialyl-Lewis x antigen-binding portions of the proteins of the invention to carbohydrates such as sialyl-Lewis x and other carbohydrate substances or carbohydrate analog substances can be used to screen combinatorial libraries of carbohydrate molecules and analogs thereof. Methods to produce combinatorial libraries and combinatorial carbohydrate or glycoconjugate libraries are well-known in the art.

The invention will now be illustrated by description of experiments and drawings, but the scope of protection is not intended to be limited to the specific disclosures.

### DESCRIPTION OF THE DRAWINGS

FIG. 1. is a diagram which shows that H. pylori strains bind the sialyl-Lewis x antigen with high affinity.

(A) H. pylori strains were analyzed for binding to ¹²⁵I-labeled neoglycoconjugates. Bacterial binding is given by the bars, from left to the right; The Lewis b-, sialyl-Lewis x-, sialyl-Lewis a-, and sialyl-α 2,3 lactose- all albumin conjugates.

(B) The 101 Swedish H. pylori strains were analyzed for neuraminidase dependent hemagglutination (HA), here shown with median values indicated in the boxplots. A strong correlation according to Pearson was found between the shift(s) in HA titers after sialidase treatment of the red blood cells (removal of sialic acid residues) and bacterial binding of the soluble ¹²⁵I-labeled sLex antigen; 0.58, p=0.000.

FIG. 2. shows the Retagging of the sialic acid binding adhesin, SabA, and identification of the corresponding gene, JHP622.

(A) The sialyl-Lewis x antigen was used with the Retagging technique for identification of the corresponding adhesin, in the babA1babA2-mutant. After contact dependent Retagging and biotin transfer, the 66 kDa biotin tagged adesin SabA, was identified by SDS-PAGE, and subjected to MALDI-TOF. As a control, the Lewis b antigen was used to Retagg the 17875 (wild type) strain, which thus visualized the 75 kDa BabA adhesin.

(B) All four peptide sequences were identified by Q-TOF and aligned with the deduced amino acid sequence of the chromosomal JHP662 gene (SEQ ID NO: 5)(4 peptide matches (two unique grey bars and the two common black bars)) and the deduced amino acid sequence of the chromosomal JHP659 gene (SEQ ID NO: 6) (2 peptide matches (the two common black bars)).

### DESCRIPTION OF EXPERIMENTS

Experimental Procedures

The procedures described herein are based on previously published teachings, and therefore the teachings of the herein cited publications are incorporated herein by reference.

Strains and Growth Conditions

H. pylori strains 26695 (Tomb et al., 1997), J99 (Alm et al., 1999), CCUG17875, and the babA-mutant stain were recently described (Ilver-Arnqvist et al., 1998). The 17875babA1::kan babA2::cam (double)-mutant strain was described in WO 00/56343. H. pylori clinical isolates were from the University Hospital in Uppsala, Sweden. Bacteria were grown at 37° C. in 10% CO₂ and 5% O₂, for 2 days.

H. pylori Binding to Neo-Glycoconjugates

¹²⁵I-labeled sialyl-α2,3lactose-, sialyl-Lewis a-, sialyl-Lewis x- and Lewis b-neoglyco-conjugates (IsoSep AB, Tullinge, Sweden) bound to bacteria were measured by gamma counting. Binding experiments were reproducible and performed in triplicates. RIA and Scatchard analyses were performed essentially as described in Ilver-Arnqvist et al., 1998.

Sialidase-Dependent Hemagglutination of H. pylori

Erythrocytes (RBC) were obtained by vein puncture from a healthy donor and were washed with PBS and used at 0.75% (v/v) concentration. Sialidase treatment of RBC was performed as described (Paulson et al, 1987) using Vibrio cholerae sialidase. Preparation of bacterial samples, titration and haemagglutination assays were performed as described before (Hirmo et al., 1996) on microtiter plates.

Purification and Identification of the SabA Adhesin by Retagging.

The SabA adhesin was purified as previously described for the BabA adhesin (Ilver-Arnqvist, et al., 1998), with some modifications. H. pylori was incubated with sialyl-Lewis x glycoconjugate, to which the Sulfo-SBED crosslinker (Pierce, Rockville, Ill.) had been conjugated, according to the manufacturers recommendations. The photo reactive crosslinker group was activated by extensive UV irradiation (12-15 hours), and then the biotin (re)tagged proteins were purified with streptavidin coated magnetic beads as described before (Ilver-Arnqvist, et al., 1998). The extracted biotin tagged proteins were then separated on SDS-PAGE, the 66 kDa band was digested with Trypsin (seq grade, Promega, U.S.A.) and analyzed on a Micromass TOF-Spec E (Micromass, Manchester, England), according to Larsson, et al., 2000. ProFound (www.proteometrics.com) was used for matching peptide masses (at NCBI). Peptide identities were validated by Q-TOF (Micromass), using the nanospray source, according to Nörregaard Jensen et al., 1999. Mascot (www.matrixscience.com) identified all our peptide sequences in the deduced amino acid sequence of JHP622 (FIG. 2 ;B) (SEQ ID NO: 5).

| | | | |
|---|---|---|---|
| SEQ ID NO: 1, | QSIQNANNIELVNSSLNYLK, | JHP622 aa 68-87 in FIG. 2;B, | Grey bar. |
| SEQ ID NO: 2, | IPTINTNYYSFLGTK, | JHP622 aa 625-639 in FIG. 2;B, | Black bar. |
| SEQ ID NO: 3, | YYGFFDYNHGYIK, | JHP622 aa 505-517 in FIG. 2;B, | Black bar. |
| SEQ ID NO: 4, | DIYAFAQNQK, | JHP622 aa 306-315 in FIG. 2;B, | Grey bar. |

Construction of the sabA-Mutant Strain

The J99 strain (Alm et al., 1999) was used for the construction of the J99sabA(JHP662)::cam- and the J99/sabB(JHP659);cam-mutant strains. The JHP662 gene was amplified using the F18 and R17 primers and cloned in pBluescript SK±EcoRV site, linearized with R20+F21 and ligated with the camR gene (Wang and Taylor, 1990). The JHP659 gene was amplified using the F16+R15 primers and cloned in pCR2.1-TOPO vector (Invitrogen, Groningen, Holland), linearized with HincII and ligated with the camR gene. The H. pylori transformants were analyzed for binding to ¹²⁵I-labeled sialyl-Lewis x glycoconjugate and the location of the camR gene in JHP662 and JHP659 was analysed using the primers R17+F18 and F16+R15, respectively, where the mutants provided larger PCR products compared to the J99-stain. The sequences of the primers are as follows:

| | | |
|---|---|---|
| R15: | CTATTCATGTTTACAATA; | SEQ ID NO: 7 |
| F16: | GGGTTTGTTGTCGCACCACTAG; | SEQ ID NO: 8 |
| R17: | GGTTCATTGTAAATATAT; | SEQ ID NO: 9 |
| F18: | CGATTCTATTAGATCACCC; | SEQ ID NO: 10 |
| R20: | AGCGTTCAATAACCCTTACAGCG; | SEQ ID NO: 11 |
| F21: | GATTTAAATACTGGCTTAATTGCTCG; | SEQ ID NO: 12 |
| BS22: | CGCTTAAAGCATTGTTGACAGCC; | SEQ ID NO: 13 |

Background Results and New Results

The Lewis b antigen binding adhesin, BabA, was recently identified (Ilver-Arnqvist et al., 1998). We then analyzed the babA-mutant strain, devoid of Lewis b antigen binding properties, for binding to human gastric mucosa, and the babA-mutant strain demonstrates an adherence pattern most comparable to the CCUG17875 parent strain (denoted 17875). Thus, we then constructed the babA1A2-(double) mutant strain, where both babA-genes were inactivated, since the tenacious adherence observed by the babA2 mutant strain could possibly have been ascribed to recombination of the remaining silent babA1 gene into expression loci. However, the adherence pattern of the babA1A2-mutant strain was still most similar to the 17975 (parent) strain. As expected, pre-treatment of the 17875 strain with soluble Lewis b antigen resulted in >80 % reduction of bacterial adherence to the epithelial cell lining. In contrast, adherence by the babA1A2-mutant strain was not affected. Screening of receptors for the babA1A2-mutant strain was performed by binding of H. pylori and mAbs to panels of glycosphingolipids (GSLs) using the thin-layer chromatogram (HPTLC) binding technique (Ångström et al., 1998). The babA1A2-mutant strain differed from the parent 17875 strain since the mutant does not recognize the Lewis b GSL. Instead, the babA1A2-mutant strain recognizes acidic GSLs from human granulocytes and adenocarcinoma cells. Binding to these GSLs was abrogated by removal of the sialic acid residues. By probing the HPTLC-plates with the sialyl-Lewis x mAb, a staining pattern almost parallel to the binding pattern of the babA1A2-mutant stain was obtained. High affinity GSLs were isolated from human adenocarcinoma tissue using the babA1A2-mutant strain as a probe. The novel H. pylori receptor, the sialyl-dimeric-Lewis x GSL demonstrated high affinity for the babA1A2-mutant strain (published in WO 00/56343).

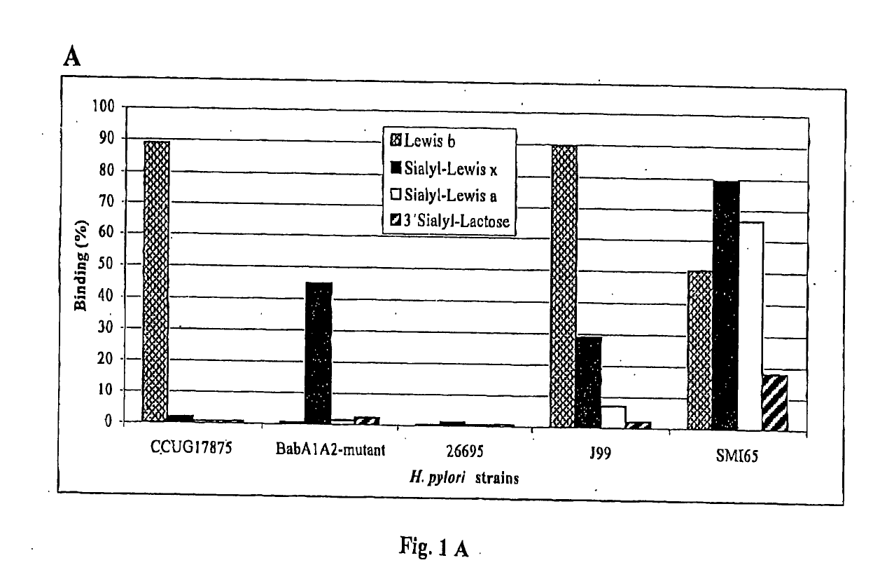

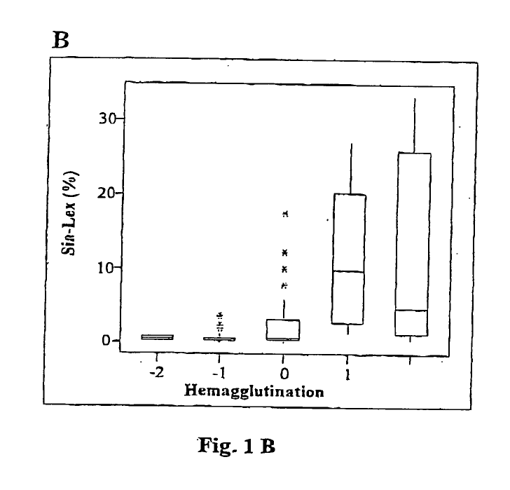

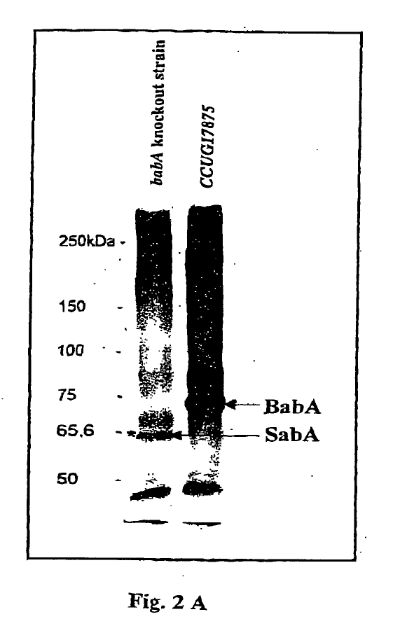

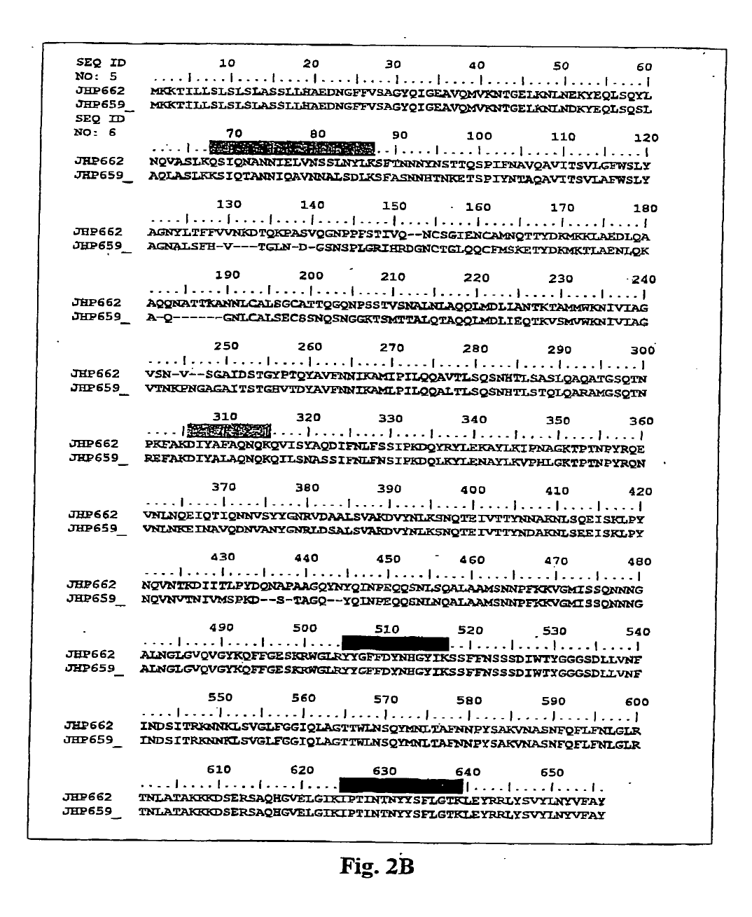

Clinical isolates of H. pylori were analyzed by binding experiments to a series of soluble semi-synthetic glycoconjugates. Several combinations of adherence modes were found where the 17875 strain binds the Lewis b antigen only, while the babA1A2-mutant strain binds sialylated antigens. In our hands, the 26695 strain (genome sequenced by Tomb et al., 1997) binds neither antigen. In contrast, the J99 strain (genome sequenced by Alm et al., 1999) recognizes both the Lewis b and the sialyl-Lewis x (sLex) antigen (FIG. 1A, and published in WO 00/56343).

The prevalence of binding to the sialyl-Lewis x antigen was assessed among Swedish clinical H. pylori isolates and 39% were found positive for binding. In comparison, 67% of the isolates bind the Lewis b antigen (Ilver-Arnqvist et al., 1998), and a majority of strains, 30 out of the 39 isolates bind both the Lewis b and the sLex antigen. Interestingly, 15 out of the 39 sLex antigen binding strains also bind the related sialyl-Lewis a antigen. (published in WO 00/56343, with small adjustments).

A Strong Correlation Found Between Sialidase Dependent Hemagglutination (HA) and Sialyl-Lewis x Antigen Binding

It has been known for more than a decade that H. pylori demonstrates sialidase dependent hemagglutination (HA), i.e. aggregation dependent on sialylated glycoconjugates on the red blood cells (Evans et al., 1988). Thus, our panel of clinical strains were subjected to HA and 27% (27/101) were found to provide positive HA-Titers. A strong correlation was found between HA titers and sialyl-Lewis x antigen binding (FIG. 1B), which suggests that previous results on HA titers of H. pylori strains, might actually relate to their ability for binding inflammation associated sLex-antigens.

Human gastric mucosa have also been analyzed for expression of sialylated glycoconjugates that promote adherence of H. pylori. Pretreatment of the babA1A2-mutant strain with the sLex conjugate abolished adherence (>90% reduction) to the gastric epithelial lining. In contrast, adherence by the 17875 parent strain was unaffected by soluble sLex conjugate. The results strongly suggest that sLex antigens promote adherence of H. pylori to the surface mucous cells in the human gastric epithelial lining (published in WO 00/56343). Non-H. pylori-infected, i.e., healthy Lewis b mouse gastric mucosa was analyzed for expression of sialylated glycoconjugates, that promote adherence of H. pylori. Pretreatment of the babA1A2-mutant strain with the sLex conjugate abolished adherence (>90% reduction) also to the Lewis b mouse gastric epithelial lining. In contrast, adherence by the 17875 parent strain was unaffected by soluble sLex conjugate. The results suggest that sLex antigens confer adherence of H. pylori to the surface mucous cells in the Lewis b mouse gastric epithelial lining (published in WO 00/56343)

Identification of the Corresponding Sialic Acid Binding Adhesin, SabA, a BabA-Related Member of the H. pylori Outer Membrane Protein (Hop) Family.

During the last decade various H. pylori proteins have been proposed as sialic acid binding adhesins or hemagglutinins (reviewed in Gerhard et al., 2001). Nevertheless, in an attempt to sort this out, we decided to identify the corresponding sLex antigen binding adhesin. Since the adhesin activity was characterized by the promising combination of high binding specificity and high affinity for the sLex antigen, our recently developed Retagging technique would be the best option for the task. Retagging is based on the use of a multifunctional biotinylated crosslinker agent chemically attached to the receptor (Ilver-Arnqvist et al., 1998). Thus, for the present Retagg experiments we used the sialyl-Lewis x conjugate. Since the affinity for the sLex antigen was lower compared to the previously described Lewis b antigen-BabA-interaction (Ilver-Arnqvist et al., 1998), the Retagging protocol was improved by use of extensive UV-exposure (see M&M). The resulting Retagging (contact dependent biotin tagging of the corresponding ligand protein) demonstrated a band of approx. 66 kDa on SDS gel (FIG. 2;A), which was analyzed by Maldi TOF. Four peptides were identified and mapped by computer analyzes to deduced amino acid sequences of the gene JHP662 in the J99 strain, but two out of the four peptides also matched the closely related deduced amino acid sequence of JHP659 (Astra/Alm et al 1999) (FIG. 2;B), i.e. the QSIQNANNIELVNSSLNYLK-peptide (grey bar in FIG. 2;B)(SEQ ID NO: 1) and the DIYAFAQNQK-peptide (grey bar in FIG. 2;B)(SEQ ID NO:4) are unique for the SabA protein (expressed by the JHP622 gene). The JHP662 and JHP659 genes are postulated outer membrane proteins with no known function. A gene knockout of JHP662 completely abrogated all binding activity for the sLex antigen. In contrast, binding activity was unperturbed by inactivation of JHP659 in the J99 strain. Thus JHP662, which corresponds to HP0725 in the 26695 strain (TIGR/Tomb et al., 1997), constitutes the gene that encodes the sialic acid binding adhesin, SabA of the present invention, while the protein encoded by the JHP659/HP0722 genes was denoted SabB.

Summary of Results

The fucosylated blood group antigens, H1 and Lewis b, mediate bacterial adherence t the stomach epithelial and mucus lining (Borén et al., 1993). We recently identified the corresponding blood group antigen binding adhesin, BabA (Ilver-Arnqvist, et al., 1998), by the Retagging technique, based on the use of multfunctionell crosslinker structures. The clinical significance of the BabA adhesin is interesting, since it is highly associated with a virulent subset of H. pylori strains, the “triple-positive” strains (Gerhard et al., 1999). The present series of experimental results are based on the use of our defined babA mutant strain, which does not bind the Lewis b antigen, but demonstrates an alternative adherence mode for targeting the gastric epithelial lining.

A high affinity glycosphingolipid (GSL) was recently identified as the sialyl-dimeric-Lewis x antigen. The prevalence of binding activity among Swedish clinical isolates was then assessed, and 39% of strains bind the sialyl-Lewis x (sLex) antigen, compared to 67% of strains that bind the Lewis b antigen. H. pylori has actually for long been known to demonstrate sialic acid dependent adhesive properties (Evans et al., 1988). Here, among the Swedish strains, 27% demonstrate such sialidase dependent hemagglutination (HA), and a strong correlation to sLex binding was found (FIG. 1;B ), which suggests that the corresponding adhesins are interchangeable or identical.

The sialyl-Lewis x and sialyl-Lewis a antigens have previously both been defined as inflammation markers and tumor antigens (Sakamoto et al., 1989; Takada et al., 1993). The binding of 15% of H. pylori strains also to the sialyl-Lewis a antigen is intriguing considering the sialyl-Lewis a antigen both a tumor antigen (Magnani et al., 1981), and gastric dysplasia marker (Sipponen et al., 1986; Farinati et al., 1988), especially in relation to H. pylori as a possible carcinogen (IARC Working Group on the Evaluation of Carcinogenic Risks to Humans, 1994). Recently, high level expression of the sialyl-dimeric-Lewis x antigen was found to correlate with poor outcome in gastric cancer (Amado et al., 1998). Blood group O phenotype and non-secretor status are independent risk factors for peptic ulcer disease (Sipponen et al., 1989). Non-secretor individuals lack the ABO blood group antigens (and the Lewis b antigen) in secretions, such as saliva, and, in addition, in the gastro-intestinal lining, where instead the Lewis a antigen and the sialyl-Lewis a antigens dominate (Sakamoto et al., 1989). In this respect it could be speculated that differences in adherence modes among H. pylori strains could promote differences in disease outcome, as a reflection of both individual blood group phenotype and secretor status.

The bacterial adherence properties were recently analyzed in relation to the mucosal inflammation response of the corresponding tissue and significant correlation was found between sLex antigen dependent adherence of the babA-mutant strain and (1) elevated levels of inflammatory cell infiltration (2) sialyl-Lewis x antigen expression, and (3) histological gastritis (published in WO 00/56343).

Recently, increased expression of the sialyl-Lewis a antigen was also demonstrated in H. pylori infected individual and the sialyl-Lewis a antigen was expressed in fewer epithelial cells after H. pylori eradication (Ota et al., 1998). Similarly, the sialyl-Lewis x antigen was found to be over-expressed in bronchial mucins from Pseudomonas aeruginosa-infected patients with chronic bronchitis (Davril et al., 1999). Thus, up-regulation of sialyl-Lewis antigens as a dynamic response to infectious agents could be a process similar to the established inflammation triggered expression of binding sites for selectin molecules in the endothelial cell lining (reviewed by Varki, 1994). In the inflamed gastric mucosa, the stimulated up-regulation of sialyl-Lewis antigen expression would then be available to H. pylori for sequential adherence modes. Thus, initial targeting to the epithelial lining by the virulent triple-positive strains would be directed by the Lewis b antigen (Gerhard et al., 1999), while the sialyl-Lewis x glycosphingolipids would mediated subsequent establishment of intimate contact with the cell membrane. Taken together, these results help out to understand the previous observations that chronic atrophic gastritis and dysplasia promote expression of sialylated structures (Sipponen et al., 1986), and that H. pylori demonstrate sialic acid dependent hemagglutination properties (Evans et al., 1988).

Here, the corresponding SabA adhesin, SEQ was purified by sialyl-Lewis x antigen primed Retagging technique, and the corresponding sabA gene was identified. The sabA gene is similar to the babA/B genes members of the Hop-family, i.e. the H. pylori outer membrane protein which all demonstrate extensive homologies in the NH₂-terminal and COOH-terminal domains (Tomb et al., 1997), where SabA and BabA demonstrate 60% similarities in the N-terminal domain, 77% similarities in the 300aa C-terminal domain, but only 32% similarities in the central region (19% identities). However, the Hop proteins were recently phylogenetically mapped on the basis of the homologous C-terminal domains, by Alm, et al., 2000. In this phyl-tree, the sabA adhesin gene (HP0725/JHP662/Hop P) and the closely related HP0722/JHP659/Hop O (in analogy denoted sabB)), map next to the Lewis b antigen binding BabA/B adhesin genes (Hop S and T, respectively), and in addition, next to the recently postulated HopZ adhesin (Alm, et al., 2000). It is tempting to speculate that the additional genes clustered in this distinct branching of the Hop-phylogeny tree constitute the adhesin repertoire of H. pylori for interaction with blood group antigen derived carbohydrates. Genetic recombination and frame shifting events would allow the easy switching on or off of adherence properties (Haas et al., 1986). Recombination within the sabA and sabB genes could also provide the potential to promote flexible presentations of adhesive modes such as adaptation to fine tuned differences in the presentation of sialylated glycoconjugates, such as affinity for the sialyl-Lewis x-versus the sialyl-Lewis a-antigens.

### REFERENCES

Alm, R A, Ling, L. S., Moir, D. T., King, B. L., Brown, E. D., Doig, P. C., Smith, D. R., Noonan, B., Guild, B. C., deJonge, B. L., et al. (1999). Genomic-sequence comparison of two unrelated isolates of the human gastric pathogen Helicobacter pylori. Nature 397, 176-180.

Alm R. A., Bina J., Andrews B. M., Doig P., Hancock R. E., Trust T. J. (2000). Comparative genomics of Helicobacter pylori: analysis of the outer membrane protein families. Infect Immun. 68, 4155-68.

Amado, M., Carneiro, F., Seixas, M., Clausen, H., and Sobrinho-Simoes, M. (1998). Dimeric sialyl-Le(x) expression in gastric carcinoma correlates with venous invasion and poor outcome. Gastroenterology 114, 462-470.

Ångström, J., Teneberg, S., Milh, M. A., Larsson, T., Leonardsson, I., Olsson, B. M., Ölwegärd-Halvarsson, M., Danielsson, D., Näslund, I., Ljungh, Å, et al. (1998). The lactosylceramide binding specificity of Helicobacter pylori. Glycobiology 8, 297-309.

Borén, T., Falk, P., Roth, K. A., Larson, G., and Normark, S. (1993). Attachment of Helicobacter pylori to human gastric epithelium mediated by blood group antigens. Science 262, 1892-1895.

Borén, T., and Falk, P. (1994). Blood type and the risk of gastric disease. Science 264, 1387-1388.

Clausen, H., and Hakomori, S. i. (1989). ABH and related histo-blood group antigens; immunochemical differences in carrier isotypes and their distribution. Vox Sang 56, 1-20.

Davril, M., Degroote, S., Humbert, P., Galabert, C., Dumur, V., Lafitte, J.1, Lamblin, G., and Roussel P. (1999). The sialylation of bronchial mucins secreted by patients suffering from cystic fibrosis or from chronic bronchitis is related to the severity of airway infection. Glycobiology 9, 311-321.

Evans, D. G., Evans, D. J., Moulds, J. J., and Graham, D. Y. (1988). N-acetylneuraminyllactose-binding fibrillar hemagglutinin of Campylobacter pylori: a putative colonization factor antigen. Infect. Immun. 56, 2896-2906.

Falk, P. G., Bry, L., Holgersson, J., and Gordon, J. I. (1995). Expression of a human -1,¾-fucosyltransferase in the pit cell lineage of FVB/N mouse stomach results in production of Le b-containing glycoconjugates: a potential transgenic mouse model for studying Helicobacter pylori infection. Proc. Natl. Acad. Sci. USA 92, 1515-1519.

Farinati F., Nitti, D., Cardin, F., Di Mario, F., Costa, F., Rossi, C., Marchett, A., Lise, M., and Naccarato, R. (1988). CA 19-9 determination in gastric juice: role in identifying gastric cancer and high risk patients. Eur. J. Cancer Clin. Oncol. 24, 923-927.

Gerhard, M., Lehn, N., Neumayer, N., Borén, T., Rad, R., Schepp, W., Miehlke, S., Classen, M., and Prinz, C. (1999). Clinical relevance of the Helicobacter pylori gene for blood-group antigen-binding adhesin. Proc. Natl. Acad Sci. USA 96, 12778-12783.

Gerhard, M., Hirmo, S., Wadström, T., Miller-Podraza, H., Teneberg, S., Karlsson, K. A., Appelmelk, B., Odenbreit, S., Haas, R, Arnqvist, A., and Borén, T. (2001). Helicobacter pylori, an adherent pain in the stomach. In Helicobacter pylori: Molecular and Cellular Biology, S. Suerbaum, and M. Achtman, eds. (Wymondham, U.K.: Horizon Scientific Press), ch. 12.

Guruge, J. L., Falk, P. G., Lorenz, R. G., Dans, M., Wirth, H. P., Blaser, M. J., Berg, D. E., and Gordon, J. I. (1998). Epithelial attachment alters the outcome of Helicobacter pylori infection. Proc. Natl. Acad. Sci. USA 95, 3925-3930.

Haas, R, and Meyer, T. F. (1986). The repertoire of silent pilus genes in Neisseria gonorrhoeae: evidence for gene conversion. Cell 44, 107-115.

Hirmo, S., Kelm, S., Schauer, R., Nilsson, B., and Wadström, T., (1996). Adhesion of Helicobacter pylori strains to α2,3-linked sialic acid. Glycoconj. J. 13, 1005-1011.

Hultgren, S. J., Abraham, S., Caparon, M., Falk, P., St Geme, J. W. 3d, and Normark S. (1993). Pilus and nonpilus bacterial adhesins: assembly and function in cell recognition. Cell 73, 887-901.

IARC Working Group on the Evaluation of Carcinogenic Risks to Humans. (1994). Schistosomes, liver flukes and Helicobacter pylori. IARC Monogr. Eval. Carcinog. Risks Hum. 61, 1-241.

Ilver, D., Arnqvist, A., Ögren, J., Frick, I. M., Kersulyte, D., Incecik, E. T., Berg, D. E., Covacci, A., Engstrand, L., and Borén, T. (1998). Helicobacter pylori adhesin binding fucosylated histo-blood group antigens revealed by Retagging. Science 279, 373-377.

Karlsson, K. A. (1998). Meaning and therapeutic potential of microbial recognition of host glycoconjugates. Mol Microbiol. 29, 1-11.

Larsson, T., Bergström, J., Nilsson, C., and Karlsson, K. A. (2000). Use of an affinity proteomics approach for the identification of low-abundant bacterial adhesins as applied on the Lewis b-binding adhesin of Helicobacter pylori. FEBS Letters 469, 155-158.

Magnani, J. L., Brockhaus, M., Smith, D. F., Ginsburg, V., Blaszczyk, M., Mitchell, K. F., Steplewski, Z., and Koprowski, H. (1981). A monosialoganglioside is a monoclonal antibody-defined antigen of coloncarcinoma. Science 212, 55-56,

Marshall, B. J., and Warren, J. R. (1984). Unidentified curved bacilli in the stomach of patients with gastritis and peptic ulceration. Lancet 1, 1311-1315.

Nörregaard-Jensen, O., Wilm, M., Shevchenko, A, and Mann, M.(1999). Peptide Sequencing of 2-DE Gel-Isolated Proteins by Nanoelectrospray Tandem Mass Spectrometry. In Methods in Molecular Biologi, 2-D Proteome Analysis Protocols, Link, A. J. ed, (Humana Press). Pp. 112, 571-588.

Ota, H., Nakayama, J., Momose, M., Hayama, M., Akamatsu, T., Katsuyama, T., Graham, D. Y., and Genta, R. M. (1998). Helicobacter pylori infection produces reversible glycosylation changes to gastric mucins. Virchows Arch 433, 419-426.

Parsonnet, J. (1998). Helicobacter pylori: the size of the problem. Gut 43, S6-S9.

Paulson, J. C., and Rogers G. N., (1987). Resialylated erythrocytes for assessment of the specificity of sialyloligosaccharide binding proteins. Methods Enzymol. 138, 162-168.

Sakamoto, J., Watanabe, T., Tokumaru, T., Takagi, H., Nakazato, H., and Lloyd, K. O. (1989). Expression of Lewis a, Lewis b, Lewis x, Lewis y, sialyl-Lewis a, and sialyl-Lewis x blood group antigens in human gastric carcinoma and in normal gastric tissue. Cancer Res. 49, 745-752.

Sipponen, P., and Lindgren, J. (1986). Sialylated Lewis a determinant CA 19-9 in benign and malignant gastric tissue. Acta Pathol. Microbiol. Immunol. Scand. 94, 305-11.

Sipponen, P., Äärynen, M., Kääriäinen, I., Kettunen, P., Helske, T., and Seppälä K. (1989). Chronic antral gastritis, Lewis a+ phenotype, and male sex as factors in predicting coexisting duodenal ulcer Scand. J. Gastroenterol. 24, 581-588.

Takada, A, Ohmori, K., Yoneda, T., Tsuyuoka, K., Hasegawa, A., Kiso, M., and Kannagi, R. (1993). Contribution of carbohydrate antigens sialyl Lewis a and sialyl Lewis x to adhesion of human cancer cells to vascular endothelium. Cancer Res. 53, 354-361.

Tomb, Jr., White, O., Kerlavage, A. R., Clayton, R. A., Sutton, G. G., Fleischmann, R. D., Ketchum, K. A., Klenk, H. P., Gill, S., Dougherty, B. A., et al. (1997). The complete genome sequence of the gastric pathogen Helicobacter pylori. Nature 388, 539-547.

Varki, A. (1994). Selectin ligands. Proc. Natl. Acad. Sci. USA 91, 7390-7397.

Wang Y, Taylor D. E., Chloramphenicol resistance in Campylobacter coli; nucleotide sequence, expression, and cloning vector construction. Gene. September 28, 1990;94(1):23-8.

WO 00/56343, Borén T. and Hammarström, L., Use of Fucosylated Sialylated N-Acetyl Lactosamine Carbohydrate Structures for Inhibition of Bacterial Adherence.

1. Isolated Helicobacter pylori protein binding to sialyl-Lewis x antigen and having an approximate molecular weight of 66 kDa and comprising the amino acid sequences

| | |
|---|---|
| SEQ ID NO: 1, | QSIQNANNIELVNSSLNYLK, |
| SEQ ID NO: 2, | IPTINTNYYSFLGTK, |
| SEQ ID NO: 3, | YYGFFDYNHGYIK, and |
| SEQ ID NO: 4, | DIYAFAQNQK, |

and sialyl-Lewis x antigen-binding H. pylori alleles of the protein, recombinant forms of the protein or the protein alleles, and sialyl-Lewis x antigen-binding portions of the proteins.

2. Protein according to claim 1, wherein the recombinant protein has the amino acid sequence SEQ ID NO: 5.

3. Protein or a sialyl-Lewis x antigen binding portion of the protein according to claim 1 or 2 for use as a medicament.

4. Diagnostic antigen for the immunological determination, in a biological sample, of antibodies against sialyl-Lewis x antigen-binding protein, wherein the diagnostic antigen is an optionally labeled protein or a sialyl-Lewis x antigen binding portion of a protein according to claim 1 or 2.

5. A method of determining the presence of sialyl-Lewis x antigen-binding H. pylori bacteria in a biological sample, which comprises an immunological determination of the presence of antibodies binding to an optionally labeled protein according to claim 1 or 2.

6. DNA molecule encoding a protein or a sialyl-Lewis x antigen binding portion of a protein according to claim 1 or 2.

7. Vector comprising a DNA molecule according to claim 6.

8. Host transformed with a vector according to claim 7.

9. Method of determining the presence of sialyl-Lewis x or related carbohydrate structures in a sample, comprising bringing the sample into contact with an optionally labelled protein or sialyl-Lewis x antigen binding portion of a protein according to claim 1 or 2, allowing binding of the protein or sialyl-Lewis x antigen binding portion of the protein according to claim 1 or 2 to the carbohydrate structure and determining the presence of sialyl-Lewis x or related carbohydrate structures in the sample by determining
a) the occurrence of the binding, or
b) the absence of binding in case an analyte inhibiting the binding is present.
